(19) United States
(12) Patent Application Publication
(10) Pub. No.: US 2012/0302731 A1
(43) Pub. Date: Nov. 29, 2012

(54) PROTEIN HYDROLYSATE, POLYPEPTIDE SOLUTION AND POLYPEPTIDE, PREPARATION METHOD AND USE THEREOF

(75) Inventors: Lin Li, Guangzhou City (CN); Bing Li, Guangzhou City (CN); Yingjuan Tian, Guangzhou City (CN); Ling Chen, Guangzhou City (CN); Liang Zhu, Guangzhou City (CN); Guoqin Liu, Guangzhou City (CN); Jianyu Su, Guangzhou City (CN); Xiaoxi Li, Guangzhou City (CN)

(73) Assignee: SOUTH CHINA UNIVERSITY OF TECHNOLOGY, (CN)

(21) Appl. No.: 13/522,306

(22) PCT Filed: Oct. 14, 2010
(86) PCT No.: PCT/CN10/77743

§ 371 Date: Jul. 13, 2012

(30) Foreign Application Priority Data
Jan. 14, 2010 (CN) 201010019415.5

(51) Int. Cl. C07K 2/00; C07K 1/34; C12P 21/06

(52) U.S. Cl. 530/344; 435/68.1; 530/300

(57) ABSTRACT

The present invention provides methods for the preparation of protein hydrolysate, peptide solution and peptide from BSG. The wet BSG or BSG powder is dispersed in extract solution to prepare the crude BSG protein or the crude BSG protein solution. Preparing the crude BSG protein solution using the crude BSG protein and adjusting the pH to 6.5˜8.5, or adjusting the pH of the crude BSG protein solution to 6.5˜8.5. Then the solution is hydrolyzed with protease at 45° C. to 65° C. for 1 h to 5 h in a water bath shaker to prepare BSG protein hydrolysate. The protein hydrolysate is heated to inactivate the protease and centrifuged to obtain the peptide solution. The peptide solution is separated by gel filtration and each peak is collected and pooled together to obtain the peptide. The protein hydrolysate, peptide solution and peptide in the present invention are all prepared from BSG which is a natural product and available at low cost throughout the year. There is no harmful material used in the production process. The results of in vitro experiment suggest that BSG peptide prepared by this method shows a significantly hypoglycemic effect.

### TECHNICAL FIELD OF THE INVENTION

The present invention relates generally to the field of food, health care product and medicine, and specifically, to protein hydrolysate, polypeptide solution and polypeptide and preparation method for the same prepared from brewers' spent grains, as well as their applications.

### BACKGROUND OF THE INVENTION

Brewer's spent grains (BSG) are one of the main abundant by-products in the brewing industry. Statistically the national annual beer production in China has reached to 30 million tons in 2010, implying that 7.5 million tons (wet weight) of BSG have been produced concomitantly. BSG is rich in protein, which accounts for around 23% to 30% of its composition on dry weight. Therefore, BSG is a good protein resource. However, for a long time the BSG is mainly used as low value cattle food or a simply land fill. And direct discharge of such products by a few manufacturers often causes environmental problem and waste of resource. Therefore, there is a growing interest in diversifying the utilization of BSG to raise economic and environmental benefits.

Bioactive peptides are defined as specific protein fragments that may impart a measurable biological effect on body functions or conditions and have potential health benefits, such as antiallergic, antihypertensive, immunomodulatory and cholesterol-lowering effects. The digestion and absorption of these peptides may be more rapid than those of amino acids. Since the bioactive peptides have pronounced physiological effect, high efficacy and low immunogenicity, they have gradually played an important role in treating human disease and become one of the most important research fields in recent years. BSG, rich in proteins, can be a good resource to prepare bioactive peptides and other high value-added products, therefore significantly improving the utilization of BSG.

Until now, utilization of BSG has been mainly limited to animal feeding. Due to the chemical composition of BSG, some researches have been carried out to find possible applications for this agro-industrial by-product. The company of Kirin Beer Kabushiki Kaisha in Japan has disclosed a process for producing protein-rich products from BSG. There was also a patent “biological protein prepared from BSG” providing a method for the preparation of biological protein from BSG. However, few researches have been carried out on the peptides and their biological activity. Although researches on biological activity of natural peptides have been conducted for many years, activities of the peptides prepared from BSG and their preparation methods have attracted little attention.

### SUMMARY OF THE INVENTION

In order to solve the problems mentioned above, an object of the present invention is to provide a method for the preparation of protein hydrolysate from BSG.

Another object of the present invention is to provide a method for the preparation of the peptide solution from BSG.

A further object of the present invention is to provide a method for the preparation of peptide from BSG.

Moreover, the present invention is to provide a kind of protein hydrolysate, peptide solution or peptide prepared from BSG implementing the method mentioned above, wherein the peptide solution or peptide has an inhibitory effect on α-glucosidase, i.e. anti-diabetic activity.

Furthermore, the present invention is to provide applications of the protein hydrolysate, peptide solution or peptide prepared from BSG mentioned above.

The objects of the present invention are achieved by the following technical solutions:
a method for the preparation of protein hydrolysate from BSG, including the following steps:

(1) The wet BSG or BSG powder is dispersed in extract solution to prepare crude BSG protein or crude BSG protein solution.

(2) Preparing crude BSG protein solution by the crude BSG protein prepared in step (1) into and adjusting the solution pH to 6.5˜8.5, or adjusting the pH of the crude BSG protein solution prepared in step (1) to 6.5˜8.5. Then the solution may be hydrolyzed with protease at 45° C. to 65° C. for 1 h to 5 h in a water bath shaker to prepare BSG protein hydrolysate.

The crude BSG protein is dissolved in buffer solution to prepare the crude BSG protein solution described in step (2). The buffer solution is disodium phosphate/citric acid buffer, disodium phosphate/potassium dihydrogen phosphate buffer, disodium phosphate/sodium dihydrogen phosphate buffer or potassium dihydrogen phosphate/sodium hydroxide buffer.

The ratio of the crude BSG protein to the buffer solution is between 1 g:10 mL (w/v) and 1 g:30 mL (w/v).

The protease used in step (2) is preferably alcalase, trypsin, flavourzyme or papain.

The ratio of the crude BSG protein to the protease is preferably between 1 g:0.1 mL (w/v) and 1 g:0.25 mL (w/v).

The extract solution used in step (1) is preferably the mixture of ethanol/sodium hydroxide or sodium carbonate/sodium bicarbonate buffer or sodium hydroxide/sodium bicarbonate buffer.

The mixture of ethanol/sodium hydroxide described above is made of ethanol and sodium hydroxide with volume ratio of 1:2. The volume concentration of the ethanol is 70% to 95% and the mole concentration of the sodium hydroxide is 0.01 to 0.10 mol/L. The pH of the sodium carbonate/sodium bicarbonate buffer and the sodium hydroxide/ sodium bicarbonate buffer is 9˜10.

The addition amount of the extract solution in step (1) is preferably 1000˜4000 mL per 100 g of BSG on dry weight. The BSG powder is dispersed in the extract solution and stirred at room temperature. The supernatant is recovered by filtration and centrifugation in order to obtain the crude BSG protein solution.

The stirring time is 60 to 120 min and the centrifugation condition is 2000 to 6000 rpm for 10 to 30 min in a refrigerated centrifuge.

The BSG powder described in step (1) is prepared by drying, crushing and screening of the wet BSG.

The preferable way of drying is freeze-drying and the powder is crushed by a universal mill and the screening is to pass through a 100 mesh sieve.

The crude BSG protein described in step (1) is preparing by adjusting the pH of the crude BSG protein solution to 4.0˜5.0, removing the supernatant and freeze-drying the precipitate.

The pH of the solution is adjusted with 0.15˜0.2 mol/L citric acid.

A method for the preparation of peptide solution from BSG, including the following steps:

The BSG protein hydrolysate prepared according to step (2) is heated to inactivate the protease and centrifuged to obtain the peptide solution.

The temperature to inactivate the protease is preferably 85° C.˜95° C. and the heating time to inactivate the protease is 5˜10 min.

The centrifugation condition is preferably 2000 to 6000 rpm for 10 to 30 min in a refrigerated centrifuge.

A method for the preparation of peptide from BSG, including the following steps:

The peptide solution prepared according to the steps described above is separated by gel filtration and each peak is collected and pooled together to obtain the peptide.

The molecular weight of the peptide is preferably between 1000 and 5000 Da.

The peptide solution is separated by a gel column Particularly, a Sephadex gel filtration chromatography column with a molecular weight separation range of 1000 Da to 5000 Da or less than 1500 Da is used as the gel column to separate the peptide solution.

The preferable feeding amount during the gel chromatography column separation is 0.2 to 0.8 g per 100 mL bed volume. The column is eluted with distilled water or neutral buffer (pH 7.0) at a flow rate of 2.0˜6.0 mL/min.

The buffer described above is disodium phosphate/citric acid buffer, disodium phosphate/potassium dihydrogen phosphate buffer, disodium phosphate/sodium dihydrogen phosphate buffer or potassium dihydrogen phosphate/sodium hydroxide buffer.

The peak with ultraviolet absorbance at 275˜285 nm is collected.

A protein hydrolysate prepared by the method described above.

A peptide solution prepared by the method described above.

A peptide prepared by the method described above.

Use of the protein hydrolysate described above for preparing an antidiabetic drug, an antidiabetic functional food or an antidiabetic health care product.

Use of the peptide solution described above for preparing an antidiabetic drug, an antidiabetic functional food or an antidiabetic health care product.

Use of the peptide described above for preparing an antidiabetic drug, an antidiabetic functional food or an antidiabetic health care product.

The antidiabetic functional food is an antidiabetic functional drink.

Use of the peptide described above for preparing an antidiabetic functional food, an antidiabetic functional drink or an antidiabetic health care product.

An antidiabetic drug made of the protein hydrolysate described above.

An antidiabetic functional food made of the protein hydrolysate described above.

An antidiabetic health care product made of the protein hydrolysate described above.

An antidiabetic drug made of the peptide solution described above.

An antidiabetic functional food made of the peptide solution described above.

An antidiabetic health care product made of the peptide solution described above.

An antidiabetic drug made of the peptide described above.

An antidiabetic functional food made of the peptide described above.

An antidiabetic health care product made of the peptide described above.

This invention is on the basis of the following principles:

1. The freeze-dried BSG is treated respectively in the following two cases. Case 1: being crushed by a universal mill to pass through a 100 mesh sieve. Case 2: being crushed by a super micro mill. The protein content of the BSG after crushing and the protein content of the extracts, which are extracted by alcohol-alkali solution from crushed BSG, are determined by Kjeldahl nitrogen method. The results indicate that the former treatment in case 1 is better than the latter in case 2. The BSG pretreatment of crushing by the universal mill and screening by the 100 mesh sieve can significantly improve the BSG protein purity of the target products to around 50%. Therefore, the invention implements this method to crush the BSG as a pretreatment for preliminary protein separation.

2. The present invention has optimized reaction conditions for the hydrolysis of BSG protein with protease. The hydrolysate obtained is analyzed for the degree of hydrolysis (DH) of BSG protein. The impacts of kinds of protease, hydrolysis temperature, hydrolysis time, BSG to buffer ratio, enzyme to substrate level and pH are investigated by the orthogonal test to optimize conditions for the hydrolysis of BSG protein with protease. The results show that the optimal hydrolysis conditions of Alcalase are as follows: pH8.0, 50° C., enzyme to substrate ratio of 0.15:1 (v/w), BSG to buffer ratio of 1:15 (w/v) and reaction time of 2 h. The further experimental results suggest that the degree of hydrolysis (DH) under the optimum conditions for enzymatic reaction was 18.54%.

The present invention has following advantages and benefits compared with prior art: The protein hydrolysate, peptide solution and peptide of the present invention are all prepared from BSG which is a natural product and available at low cost throughout the year. There is no harmful material used in the production process. The results of in vitro experiment suggest that BSG peptide prepared by this method shows significant hypoglycemic effect, which can be added to food as functional component for antidiabetic effect.

### BRIEF DESCRIPTION OF THE DRAWINGS

FIG. 1 shows the process flow diagram of the present invention.

FIG. 2 shows the elution curve of crude peptide obtained by gel filtration with Sephadex G15 in the 2^{nd} embodiment.

### DETAILED DESCRIPTION OF PREFERRED EMBODIMENTS

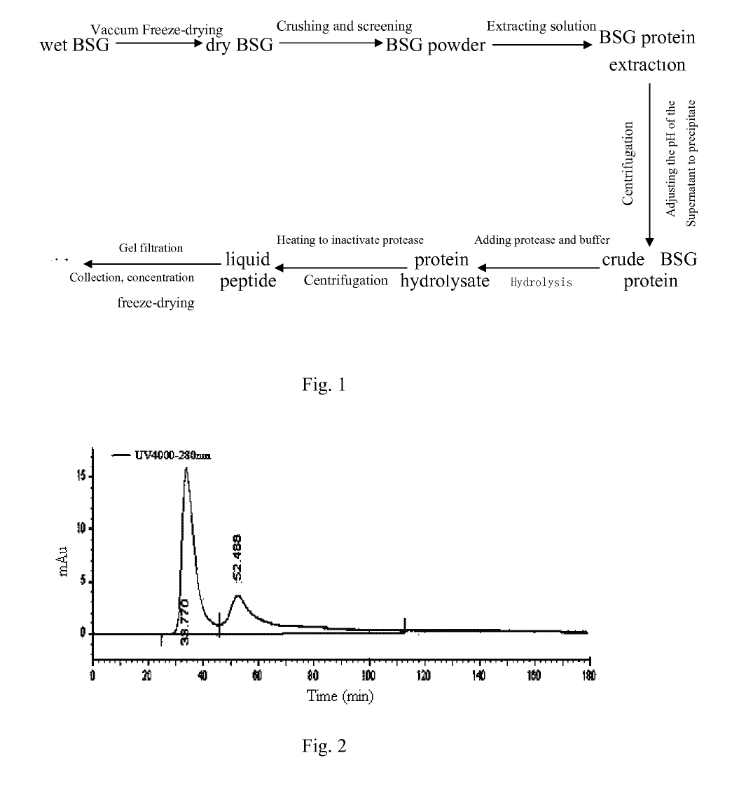

The present invention will be more specifically described by way of embodiments and accompanying drawings, by which other purposes, features and advantages of the present invention will become more obvious. However, the present invention is not limited by the given examples and embodiments, and the combination of features described in the embodiments is not always necessary features of the present invention. In the following details, FIG. 1 is the process flow diagram and FIG. 2 shows the elution curve of crude peptide obtained by gel filtration with Sephadex G15.

### The 1^{st} Embodiment (the Process Flow Diagram is as Shown in FIG. 1)

(1) The wet BSG is freeze-dried and crushed by a universal mill to pass through a 100 mesh sieve to obtain dry BSG for further use. Per 100 gram of BSG is dispersed in 1000 mL of extract solution in a ratio of 1:10 (w:v). The extract solution is a mixture of 95% ethanol and 0.01 mol/L sodium hydroxide with volume ratio of 1:2 (v:v). The solution is extracted by stirring for 60 min at room temperature and filtered, and centrifuged at 2000 rpm for 30 min to obtain the protein extraction. Adjust the pH of the supernatant for the isoelectric point precipitation of the protein (pH 4.5) with 0.2 mol/L citric acid. The supernatant is removed by centrifugation and the precipitate is freeze-dried to obtain the crude BSG protein.

(2) The crude BSG protein prepared in step (1) is dissolved in disodium phosphate/citric acid buffer. Adjust the pH of the solution to 6.5. The mixture is hydrolyzed with Alcalase at 65° C. for 1 h in a water bath shaker to prepare protein hydrolysate. The ratios of the crude BSG protein to buffer solution and the crude BSG protein to protease are 1 g:10 mL (w/v) and 1 g:0.15 mL (w/v) respectively.

(3) The protein hydrolysate prepared in step (2) is heated to 95° C. for 5 min to inactivate the Alcalase, and then centrifuged at 2000 rpm for 30 min to obtain the peptide solution.

(4) The peptide solution prepared in step (3) is separated and desalted by a Sephadex gel filtration chromatography column with a molecular weight separation range of less than 1500 Da. The sample feeding amount during the gel filtration chromatography separation is 0.2 g per 100 mL bed volume. The column is eluted with distilled water at a flow rate of 2.0 mL/min. Collect the eluted solution with ultraviolet absorbance at 275 nm to obtain the bioactive peptide. The collected fractions are then concentrated and freeze-dried in usual way to prepare bioactive peptide powder.

The results of hypoglycemic activity test indicate that the bioactive peptide could significantly inhibit α-glucosidase, which means the bioactive peptide may have hypoglycemic effect. The inhibitory activity reaches the maximum of 45.9% when the concentration of the peptide is 0.3 mg/mL and the concentration of the sucrose is 0.1 mol/L.

The protein hydrolysate, peptide solution and bioactive peptide prepared by this method can be widely used in the production of food, drinks, drugs and health care products. The bioactive peptide with hypoglycemic effect can be widely used to produce antidiabetic drugs.

### The 2^{nd} Embodiment

(1) The wet BSG is freeze-dried and crushed by a universal mill to pass through a 100 mesh sieve to obtain dry BSG for further use. Per 100 gram of BSG is dispersed in 4000 mL of extract solution in a ratio of 1:40. The extract solution is sodium carbonate-sodium bicarbonate buffer (pH 9.0). The solution is extracted by stirring for 120 min at room temperature and filtered, and centrifuged at 6000 rpm for 10 min to obtain the protein extraction. Adjust the pH of the supernatant for the isoelectric point precipitation of the protein (pH 4.0) with 0.3 mol/L citric acid. The supernatant is removed by centrifugation and the precipitate is freeze-dried to obtain the crude BSG protein.

(2) The crude BSG protein prepared in step (1) is dissolved in disodium phosphate/potassium dihydrogen phosphate buffer. Adjust the pH of the solution to 8.5. The mixture is hydrolyzed with trypsin at 45° C. for 2 h in a water bath shaker to prepare protein hydrolysate. The ratios of the crude BSG protein to buffer solution and the crude BSG protein to protease are 1 g:20 mL (w/v) and 1 g:0.25 mL (w/v) respectively.

(3) The protein hydrolysate prepared in step (2) is heated to 85° C. for 10 min to inactivate the trypsin, and then centrifuged at 6000 rpm for 10 min to obtain the peptide solution.

(4) The peptide solution prepared in step (3) is separated and desalted by a Sephadex gel filtration chromatography column with a molecular weight separation range of less than 1500 Da, The sample feeding amount during the gel filtration chromatography separation is 0.6 g per 100 mL bed volume. The column is eluted with disodium phosphate/citric acid buffer (pH 7.0) at a flow rate of 3.0 mL/min Collect the eluted solution with ultraviolet absorbance at 280 nm to obtain the bioactive peptide. The collected fractions are then concentrated and freeze-dried in usual way to prepare bioactive peptide powder. The elution curve of the crude peptide solution obtained by gel filtration with Sephadex G15 is as shown in FIG. 2. According to detection results of a UV detector, there are two absorbance peaks at 280 nm, named peak I (the elution time is 33.8 min to 52.5 min) and peak II (the elution time is 52.5 min to 115.0 min).

The results of hypoglycemic activity test indicate that the bioactive peptide could significantly inhibit α-glucosidase, which means the bioactive peptide may have hypoglycemic effect. The inhibitory activity is 30.8% when the concentrations of the peptide and the sucrose are 0.2 mg/mL and 0.1 mol/L, respectively.

The protein hydrolysate, peptide solution and bioactive peptide prepared by this method can be widely used in the production of food, drinks, drugs and health care products. The protein hydrolysate, peptide solution and bioactive peptide with hypoglycemic effect can be widely used to produce health care products which may inhibit the increase of blood glucose.

### The 3^{rd} Embodiment

(1) Per 100 gram of dry BSG is dispersed in 3000 mL of extract solution in a ratio of 1:30. The extract solution is sodium hydroxide-sodium bicarbonate buffer (pH 10.0). The solution is extracted by stirring for 70 min at room temperature and filtered, and centrifuged at 4000 rpm for 20 min to obtain the protein extraction. Adjust the pH of the supernatant for the isoelectric point precipitation of the protein (pH 5.0) with 0.15 mol/L citric acid. The supernatant is removed by centrifugation and the precipitate is freeze-dried to obtain the crude BSG protein.

(2) The crude BSG protein prepared in step (1) is dissolved in disodium phosphate/sodium dihydrogen phosphate buffer. Adjust the pH of the solution to 7.5. The mixture is hydrolyzed with flavourzyme at 50° C. for 5 h in a water bath shaker to prepare protein hydrolysate. The ratios of the crude BSG protein to buffer solution and the crude BSG protein to protease are 1 g:30 mL (w/v) and 1 g:0.10 mL (w/v) respectively.

(3) The protein hydrolysate prepared in step (2) is heated to 90° C. for 8 min to inactivate the flavourzyme, and then centrifuged at 3000 rpm for 15 min to obtain the peptide solution.

(4) The peptide solution prepared in step (3) is separated and desalted by a Sephadex gel filtration chromatography column with a molecular weight separation range from 1000 Da to 5000 Da, The sample feeding amount during the gel filtration chromatography separation is 0.8 g per 100 mL bed volume. The column is eluted with disodium phosphate/potassium dihydrogen phosphate buffer (pH 7.0) at a flow rate of 4.0 mL/min. Collect the eluted solution with ultraviolet absorbance at 285 nm to obtain the bioactive peptide. The collected fractions are then concentrated and freeze-dried in usual way to prepare bioactive peptide powder.

The results of hypoglycemic activity test indicate that the bioactive peptide could significantly inhibit α-glucosidase, which means the bioactive peptide may have hypoglycemic effect. The inhibitory activity is 40.3% when the concentrations of the peptide and the sucrose are 0.3 mg/mL and 0.15 mol/L, respectively.

The protein hydrolysate, peptide solution and bioactive peptide prepared by this method can be widely used in the production of food, drinks, drugs and health care products. The protein hydrolysate, peptide solution and bioactive peptide with hypoglycemic effect can be widely used to produce antidiabetic functional drinks.

### The 4^{th} Embodiment

(1) Per 100 gram of dry BSG is dispersed in 2000 mL of extract solution in a ratio of 1:20. The extract solution is a mixture of 70% ethanol and 0.08 mol/L sodium hydroxide with volume ratio of 1:2 (v:v). The solution is extracted by stirring for 80 min at room temperature and filtered, and centrifuged at 3000 rpm for 15 min to obtain the protein extraction. Adjust the pH of the supernatant for the isoelectric point precipitation of the protein (pH 4.7) with 0.25 mol/L citric acid. The supernatant is removed by centrifugation and the precipitate is freeze-dried to obtain the crude BSG protein.

(2) The crude BSG protein prepared in step (1) is dissolved in potassium dihydrogen phosphate/ sodium hydroxide buffer. Adjust the pH of the solution to 7.0. The mixture is hydrolyzed with papain at 55° C. for 3 h in a water bath shaker to prepare protein hydrolysate. The ratios of the crude BSG protein to buffer solution and the crude BSG protein to protease are 1 g:25 mL (w/v) and 1 g:0.20 mL (w/v) respectively.

(3) The protein hydrolysate prepared in step (2) is heated to 88° C. for 7 min to inactivate the papain, and then centrifuged at 4000 rpm for 20 min to obtain the peptide solution.

(4) The peptide solution prepared in step (3) is separated and desalted by a Sephadex gel filtration chromatography column with a molecular weight separation range from 1000 Da to 5000 Da, The sample feeding amount during the gel filtration chromatography separation is 0.3 g per 100 mL bed volume. The column is eluted with disodium phosphate/sodium dihydrogen phosphate buffer (pH 7.0) at a flow rate of 8.0 mL/min. Collect the eluted solution with ultraviolet absorbance at 282 nm to obtain the bioactive peptide. The collected fractions are then concentrated and freeze-dried in usual way to prepare bioactive peptide powder.

The results of hypoglycemic activity test indicate that the bioactive peptide could significantly inhibit α-glucosidase, which means the bioactive peptide may have a hypoglycemic effect. The inhibitory activity reaches the maximum of 42.3% when the concentrations of the peptide and the sucrose are 0.3 mg/mL and 0.1 mol/L, respectively.

The protein hydrolysate, peptide solution and bioactive peptide prepared by this method can be widely used in the production of food, drinks, drugs and health care products. The bioactive peptide with hypoglycemic effect can be widely used to produce antidiabetic drugs.

### The 5^{th} Embodiment

(1) The wet BSG is freeze-dried and crushed by a universal mill to pass through a 100 mesh sieve for further use. Per 100 gram of BSG is dispersed in 2500 mL of extract solution in a ratio of 1:25. The extract solution is sodium hydroxide-sodium bicarbonate buffer (pH 9.0). The solution is extracted by stirring for 90 min at room temperature and filtering, and centrifuged at 2800 rpm for 14 min to obtain the protein extraction. Adjust the pH of the supernatant for the isoelectric point precipitation of the protein (pH 4.6) with 0.22 mol/L citric acid. The supernatant is removed by centrifugation and the precipitate is freeze-dried to obtain the crude BSG protein.

(2) The crude BSG protein prepared in step (1) is dissolved in disodium phosphate/citric acid buffer. Adjust the pH of the solution to 7.2. The mixture is hydrolyzed with Alcalase at 57° C. for 4 h in a water bath shaker to prepare protein hydrolysate. The ratios of the crude BSG protein to buffer solution and the crude BSG protein to protease are 1 g:15 mL (w/v) and 1 g:0.18 mL (w/v) respectively.

(3) The protein hydrolysate prepared in step (2) is heated to 82° C. for 8 min to inactivate the Alcalase, and then centrifuged at 5000 rpm for 25 min to obtain the peptide solution.

(4) The peptide solution prepared in step (3) is separated and desalted by a Sephadex gel filtration chromatography column with a molecular weight separation range of less than 1500 Da. The sample volume injected into the gel filtration chromatography column is 0.5 g per 100 mL bed volume. The column is eluted with potassium dihydrogen phosphate/sodium hydroxide buffer (pH 7.0) at a flow rate of 6.0 mL/min. Collect the eluted solution with ultraviolet absorbance at 277 nm to obtain the bioactive peptide. The collected fractions are then concentrated and freeze-dried in usual way to prepare bioactive peptide powder.

The results of hypoglycemic activity test indicated that the bioactive peptide could significantly inhibit α-glucosidase, which means the bioactive peptide may have hypoglycemic effect. The inhibitory activity reaches the maximum of 41.6% when the concentrations of the peptide and the sucrose are 0.3 mg/mL and 0.1 mol/L, respectively.

The protein hydrolysate, peptide solution and bioactive peptide prepared by this method can be widely used in the production of food, drinks, drugs and health care products. The bioactive protein hydrolysate, peptide solution and peptide with hypoglycemic effect can be used to produce an antidiabetic functional drink.

### Example 6

The hypoglycemic effect of the BSG peptide prepared in the method described above is investigated by in vitro tests. The results indicate that the BSG peptide with certain peptide concentration prepared in this method has significant hypoglycemic effect. The test method described herein for evaluating hypoglycemic effect is as follow:

1. The Method for Evaluating Hypoglycemic Effect

1.1 Assay for α-Glucosidase Activity

The reaction mixture contains 0.6 mL potassium phosphate buffer (pH 6.8) and 0.1 mL α-glucosidase solution and 0.1 mL sucrose solution. These solutions are incubated at 37° C. for 10 min and the reaction is terminated by adding 1 mL of 0.1 mol/L Na₂CO₃. Glucose content is measured by a glucose assay kit. A lower glucose threshold of 5.55 mmol/L is defined as standard control. A α-glucosidase activity unit is defined as 1 μmol glucose generated per min in 1 liter of reaction mixture at 37° C. and pH 6.8.

1.2 The Effect of the BSG Peptide on α-Glucosidase Activity

The reaction mixture contains 0.3 mL peptide solution purified by gel filtration, 0.6 mL of potassium phosphate buffer (pH 6.8) and 0.1 mL of enzyme solution. The control is added the same volume of distilled water instead of peptide solution. The reaction mixture is incubated in the water bath at 37° C. for 10 min Other steps are the same as the assay for α-glucosidase activity described above. The α-glucosidase inhibitory activity of the BSG peptide is expressed as inhibition (%) and calculated as follow: inhibition (%)=(E_{control}−Eₛₐₘₚₗₑ)/E_{control}×100%, where E_{control} is the measured enzyme activity of the control mixture, and Eₛₐₘₚₗₑ is the measured enzyme activity of the peptide sample.

2. The Results of Hypoglycemic Activity Test

The results of hypoglycemic effect in vitro experiment suggest that the two peaks described as Peak I and Peak II collected from gel filtration chromatography column have inhibitory effect on α-glucosidase, or anti-diabetic activity. Results on the influence of peptide concentration on the hypoglycemic effect suggest that the inhibitory activity on α-glucosidase of the peptide first rapidly increases to a maximum and then decreases with the increase of BSG peptide concentration. It is shown that a strong inhibitory effect on α-glucosidase appears at the peptide concentration ranging from 0.2 mg/mL to 0.4 mg/mL. At the same time, the inhibition of α-glucosidase activity by BSG peptide of 0.3 mg/mL decreases with the increase of the sucrose concentration which is used as the substrate. The inhibitory activity of the BSG peptide on α-glucosidase reaches the maximum of 45.85% when the concentration of sucrose is 0.1 mol/L and the minimum of 5.56% when the concentration of sucrose is 0.35 mol/L.

Therefore, the BSG peptide prepared in this method can be widely used in the production of food, drugs and health care products with significiant antidiabetic effect.

Although the foregoing invention has been described in detail by way of illustration and example for purposes of clarity of understanding, it is readily apparent to those skilled in the art in light of the teachings of this invention that certain changes and modifications may be made thereto without departing from the spirit or scope of the appended claims.

1. A method for the preparation of a peptide solution from BSG, which comprises the following steps:
(1) the wet BSG or BSG powder is dispersed in extract solution to prepare crude BSG protein or crude BSG protein solution;
(2) preparing the crude BSG protein solution by the crude BSG protein prepared in step (1) into and adjusting the pH to 6.5˜8.5, or adjusting the pH of the crude BSG protein solution prepared in step (1) to 6.5˜8.5., then the solution is hydrolyzed with protease at 45° C. to 65° C. for 1 h to 5 h in a water bath shaker to prepare BSG protein hydrolysate; and
(3) inactivating the proteases in the BSG protein hydrolysate prepared in step (2), then centrifugate the hydrolysate to obtain the peptide solution.

2. The method according to claim 1, wherein the crude BSG protein solution described in step (2) is prepared by dissolving the crude BSG protein in a buffer solution, wherein the buffer solution is disodium phosphate/ citric acid buffer, disodium phosphate/potassium dihydrogen phosphate buffer, disodium phosphate/sodium dihydrogen phosphate buffer, or potassium dihydrogen phosphate/ sodium hydroxide buffer, wherein the ratio of the crude BSG protein to the buffer solution is between 1 g:10 mL (w/v) and 1 g:30 mL (w/v), wherein the protease described in step (2) is alcalase, trypsin, flavourzyme, or papain, and wherein the ratio of the crude BSG protein to the protease is between 1 g:0.1 mL (w/v) and 1 g:0.25 mL (w/v).

3-5. (canceled)

6. The method according to claim 1, wherein the BSG powder described in step (1) is prepared by drying, crushing and screening of the wet BSG, wherein the crude BSG protein described in step (1) is precipitated by adjusting the pH of the crude BSG protein solution to 4.0˜5.0 using 0.15˜0.2 mol/L citric acid, removing the supernatant and freeze-drying, wherein the extract solution used in step (1) is the mixture of ethanol/sodium hydroxide or sodium carbonate/sodium bicarbonate buffer or sodium hydroxide/sodium bicarbonate buffer, wherein the addition amount of the extract solution in step (1) is 1000˜4000 mL per 100 g of BSG on dry weight, and the BSG powder is dispersed in the extract solution and stirred for 60 to 120 min at room temperature, and wherein the supernatant is recovered by filtrating and centrifuging at 2000 to 6000 rpm for 10 to 30 min in a refrigerated centrifuge to obtain the crude BSG protein hydrolysate.

7. The method according to claim 6, wherein the mixture of ethanol/sodium hydroxide is made of ethanol and sodium hydroxide with volume ratio of 1:2, and the volume concentration of the ethanol is 70% to 95% and the mole concentration of the sodium hydroxide is 0.01 to 0.10 mol/L, and the pH of the sodium carbonate/sodium bicarbonate buffer and the sodium hydroxide/ sodium bicarbonate is 9˜10.

8-14. (canceled)

15. The method according to claim 1, wherein the temperature to inactivate the protease described in step (3) is 85° C.˜95° C. and the heating time to inactivate the protease is 5˜10 min.

16. (canceled)

17. A method for the preparation of peptide from BSG, which comprises the following steps:
the peptide solution prepared according to claim 1 is separated by gel filtration, and
each peak is collected and pooled together to obtain the peptide.

18. The method according to claim 17, wherein the molecular weight of the peptide is between 1000 and 5000 Da, wherein the peptide solution is separated by a sephadex gel filtration chromatography column with a molecular weight separation range of 1000 Da to 5000 Da or less than 1500 Da, wherein the peak with ultraviolet absorbance at 275˜285 nm is collected.

19. (canceled)

20. The method according to claim 18, wherein the sample feeding amount during the gel filtration chromatography separation is 0.2 to 0.8 g per 100 mL bed volume, and the column is eluted with distilled water or neutral buffer (pH 7.0) at a flow rate of 2.0˜6.0 mL/min, wherein the buffer is disodium phosphate/citric acid buffer, disodium phosphate/potassium dihydrogen phosphate buffer, disodium phosphate/sodium dihydrogen phosphate buffer, or potassium dihydrogen phosphate/sodium hydroxide buffer.

21-23. (canceled)

24. A peptide solution prepared by the method according to claim 1.

25. A peptide prepared by the method according to claim 17.

26-27. (canceled)

28. Use of the peptide solution described in claim 24 for preparing an antidiabetic drug, an antidiabetic functional food or an antidiabetic health care product.

29. (canceled)

30. Use of the peptide described in claim 25 for preparing an antidiabetic drug, an antidiabetic functional food or an antidiabetic health care product.

31-34. (canceled)

35. An antidiabetic drug made of the peptide solution described in claim 24.

36. An antidiabetic functional food made of the peptide solution described in claim 24.

37. An antidiabetic health care product made of the peptide solution described in claim 24.

38. An antidiabetic drug made of the peptide described in claim 25.

39. An antidiabetic functional food made of the peptide described in claim 25.

40. An antidiabetic health care product made of the peptide described in claim 25.
